(19) United States
(12) Patent Application Publication
(10) Pub. No.: US 2024/0420587 A1
(43) Pub. Date: Dec. 19, 2024

(54) DRUG KNOWLEDGE QUIZ METHOD, APPARATUS, ELECTRONIC DEVICE AND MEDIUM

(71) Applicant: BEIJING BAIDU NETCOM SCIENCE TECHNOLOGY CO., LTD., Beijing (CN)

(72) Inventors: Zhenhui SHI, Beijing (CN); Yuan XIA, Beijing (CN); Jun CHEN, Beijing (CN); Haifeng HUANG, Beijing (CN)

(73) Assignee: BEIJING BAIDU NETCOM SCIENCE TECHNOLOGY CO., LTD., Beijing (CN)

(21) Appl. No.: 18/749,421

(22) Filed: Jun. 20, 2024

(30) Foreign Application Priority Data
Mar. 4, 2024 (CN) 202410245432.2

(51) Int. Cl. G09B 7/02

(52) U.S. Cl. CPC G09B 7/02

(57) ABSTRACT

A method includes: performing retrieval in question-answer text in a drug knowledge base based on drug question text, to obtain answer text corresponding to the drug question text; performing retrieval in drug instructions in the drug knowledge base based on the drug question text, to obtain instruction text corresponding to the drug question text; and generating response text corresponding to the drug question text based on the answer text and the instruction text.

### CROSS-REFERENCE TO RELATED APPLICATION

The present application claims priority to Chinese Patent Application No. 2024102454322 filed on Mar. 4, 2024, which is incorporated herein by reference in its entirety.

### TECHNICAL FIELD

The present disclosure relates to the technical field of artificial intelligence, specifically to the technical fields of computer vision, image processing, deep learning, large language models, smart healthcare, etc., and in particular to a drug knowledge question answering method and apparatus, an electronic device, and a storage medium.

### BACKGROUND ART

The professional and rigorous description for current paper instructions for drugs is sometimes too complex for ordinary consumers, making it difficult for the consumers to understand practical use conditions for the drugs. This causes bother to patients or may lead to errors or misunderstandings during use of the drugs in some cases. In addition, instructions for some drugs include much text content having a large length, which takes a lot of time for readers to read. In the fast-paced modern living environment, many users cannot afford to spend much time reading complex and lengthy drug instructions, which may cause them to overlook important information about the use of the drugs. Meanwhile, textual description of some content is omitted in existing drug instructions, making information received by users incomplete, which may lead to misunderstandings about the drugs and affect the correct use of the drugs.

### SUMMARY OF THE INVENTION

The present disclosure provides a drug knowledge question answering method and apparatus, an electronic device, and a storage medium.

According to a first aspect of the present disclosure, there is provided a drug knowledge question answering method. The method includes:
obtaining drug question text;
performing retrieval in question-answer text in a drug knowledge base based on the drug question text, to obtain answer text corresponding to the drug question text;
performing retrieval in drug instructions in the drug knowledge base based on the drug question text, to obtain instruction text corresponding to the drug question text; and
generating response text corresponding to the drug question text based on the answer text and the instruction text.

According to a second aspect of the present disclosure, there is provided an electronic device. The electronic device includes:
at least one processor; and
a memory communicatively connected to the at least one processor, where
the memory stores instructions executable by the at least one processor, and the instructions, when executed by the at least one processor, cause the at least one processor to perform the following steps:
obtaining drug question text;
performing retrieval in question-answer text in a drug knowledge base based on the drug question text, to obtain answer text corresponding to the drug question text;
performing retrieval in drug instructions in the drug knowledge base based on the drug question text, to obtain instruction text corresponding to the drug question text; and
generating response text corresponding to the drug question text based on the answer text and the instruction text.

According to a third aspect of the present disclosure, there is provided a non-transitory computer-readable storage medium storing computer instructions. The computer instructions are used to cause a computer to perform the following steps:
obtaining drug question text;
performing retrieval in question-answer text in a drug knowledge base based on the drug question text, to obtain answer text corresponding to the drug question text;
performing retrieval in drug instructions in the drug knowledge base based on the drug question text, to obtain instruction text corresponding to the drug question text; and
generating response text corresponding to the drug question text based on the answer text and the instruction text.

It should be understood that the content described in this section is not intended to identify critical or important features of the embodiments of the present disclosure, and is not used to limit the scope of the present disclosure. Other features of the present disclosure will be readily understood with reference to the following description.

### BRIEF DESCRIPTION OF THE DRAWINGS

The accompanying drawings are used for a better understanding of the solutions, and do not constitute a limitation on the present disclosure. In the accompanying drawings:

FIG. 1 is a schematic diagram of steps of a drug knowledge question answering method according to an embodiment of the present disclosure;

FIG. 2 is a schematic diagram of steps of another drug knowledge question answering method according to an embodiment of the present disclosure;

FIG. 3 is a schematic diagram of specific steps of S103 of a drug knowledge question answering method according to an embodiment of the present disclosure;

FIG. 4 is a schematic diagram of specific steps of S1033 of a drug knowledge question answering method according to an embodiment of the present disclosure;

FIG. 5 is a schematic diagram of an overall process of evidence retrieval according to an embodiment of the present disclosure;

FIG. 6 is a schematic diagram of specific steps of S104 of a drug knowledge question answering method according to an embodiment of the present disclosure;

FIG. 7 is a schematic block diagram of a drug knowledge question answering apparatus according to an embodiment of the present disclosure;

FIG. 8 is a schematic block diagram of another drug knowledge question answering apparatus according to an embodiment of the present disclosure;

FIG. 9 is a schematic block diagram of a second retrieval module according to an embodiment of the present disclosure;

FIG. 10 is a schematic block diagram of a text extraction unit according to an embodiment of the present disclosure;

FIG. 11 is a schematic block diagram of a generation module according to an embodiment of the present disclosure; and

FIG. 12 is a block diagram of an electronic device for implementing a drug knowledge question answering method according to an embodiment of the present disclosure.

### DETAILED DESCRIPTION OF EMBODIMENTS

Exemplary embodiments of the present disclosure are described below in conjunction with the accompanying drawings, where various details of the embodiments of the present disclosure are included to facilitate understanding, and should only be considered as exemplary. Therefore, those of ordinary skill in the art should be aware that various changes and modifications can be made to the embodiments described herein, without departing from the scope and spirit of the present disclosure. Likewise, for clarity and conciseness, the description of well-known functions and structures is omitted in the following description.

In a current first type of drug knowledge question answering system, an answer usually is manually given by a specialist or medical professional, i.e., a user asks a question in the question answering system, the system assigns the question to different experts, the experts write answers, and then the system returns the answers to the user. Such a question answering system requires a number of specialists or medical professionals, leading to a high labor cost. The problem such as an inefficient or untimely artificial response results in a less responsive system, causing poor user experience.

In a current second type of drug knowledge question answering system, an answer is given based on a question answering library, where a user asks a question in the question answering system, and then the system matches the question of the user with a question in the question answering library; if the match succeeds, an answer corresponding to the question in the question answering library is returned to the user; and if the match fails, a default answer is given or an answer is manually given by a specialist or medical professional. Therefore, it is required to mine the question answering system in advance or manually organize a batch of questions and answers, so as to construct a question answering library, leading to a high labor cost. The effect of an algorithm for matching the question of the user with the question in the question answering library is poor, resulting in low answering accuracy and low recall.

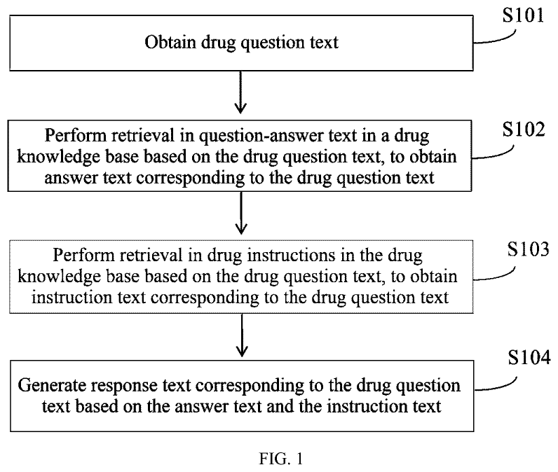

In view of the above technical problem, the present disclosure provides a drug knowledge question answering method. As shown in FIG. 1, the method includes the following steps.

Step S101: Obtain drug question text.

Step S102: Perform retrieval in question-answer text in a drug knowledge base based on the drug question text, to obtain answer text corresponding to the drug question text.

Step S103: Perform retrieval in drug instructions in the drug knowledge base based on the drug question text, to obtain instruction text corresponding to the drug question text.

Step S104: Generate response text corresponding to the drug question text based on the answer text and the instruction text.

Specifically, the drug question text in this embodiment refers to a query entered by the user, such as a question “What is the best drug for treating headaches?”, “What are the side effects of this drug?”, etc. The question-answer text refers to QA pairs, where each QA pair includes a question and a corresponding answer. For example, for a question “What are the side effects of ibuprofen?”, a corresponding answer is “The side effects of ibuprofen may be gastrointestinal discomfort including nausea, vomiting, abdominal pain, burning sensation or mild indigestion, or gastrointestinal ulcers and bleeding, and sometimes may cause a rash, urticaria, etc.”. In step S102, QA pair matching may be performed first, and if a corresponding question Q is found, a corresponding answer A is directly returned as the answer text.

If no answer text is found in step S102, drug instruction matching may be performed in step S103 to find a drug instruction corresponding to a drug name contained in the query, and then related drug knowledge is retrieved from the corresponding drug instruction and returned to the user. The drug instruction usually includes much content, and the retrieval may be performed to return only drug knowledge relevant to the query to the user. For example, if the user enters “What are the side effects of this drug?”, only a paragraph relevant to the side effects in the drug instruction is to be extracted as the instruction text, so as to implement accurate matching.

The present disclosure allows a combination of the retrieval in the question-answer text and retrieval in drug instruction paragraphs, where the answer related to the user query may be quickly and accurately retrieved by the retrieval in the question-answer text, and when no accurate answer is found, the retrieval in the drug instruction paragraphs may be performed as a supplement, so as to ensure the recall of the question answering system, improving the answering accuracy and the recall of the system.

As an optional implementation, in step S102 of performing retrieval in question-answer text in a drug knowledge base based on the drug question text, to obtain answer text corresponding to the drug question text, a conventional retrieval algorithm, such as a TF-IDF algorithm, may be applied. Alternatively, a vector retrieval method may be applied, where most relevant content is recalled by calculating cosine similarity etc. between a user query vector and a drug instruction content vector. For example, similarity is calculated between a query and a question Q in a QA library, then QA pairs are sorted according to similarity scores, and QA pairs with top 5 similarity scores are selected, thereby implementing the accurate matching for the question of the user.

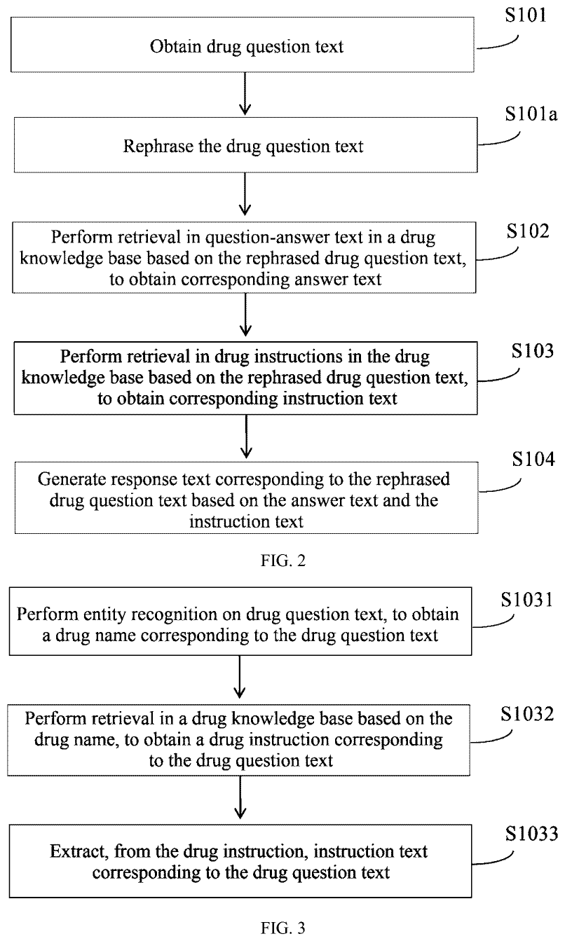

As an optional implementation, before step S102 of performing retrieval in question-answer text in a drug knowledge base based on the drug question text, as shown in FIG. 2, the method further includes the following step:

Step S101a: Rephrase the drug question text.

Specifically, as shown in FIG. 2, the drug knowledge question answering method in this embodiment includes the following steps:

Step S101: Obtain the drug question text.

Step S101a: Rephrase the drug question text.

Step S102: Perform retrieval in the question-answer text in the drug knowledge base based on the rephrased drug question text, to obtain corresponding answer text.

Step S103: Perform retrieval in the drug instructions in the drug knowledge base based on the rephrased drug question text, to obtain corresponding instruction text.

Step S104: Generate response text corresponding to the rephrased drug question text based on the answer text and the instruction text.

Users may use various languages and expressions to describe their needs during queries for drug information. For example, the query may be “What is the best drug for treating headaches?”, “What are the side effects of this drug?”, etc. In a classical natural language processing technology, it is required to perform an operation, such as word segmentation or stop word removal, on the user query, or to rephrase the user query according to a preset rule. In this embodiment, the above operation may be implemented with the help of a prompting technique of a large language model (LLM). Specifically, contextual historical dialog information of user queries and a current question entered by the user are assembled into a complete contextual dialog, and then dialog content and a prompt requirement are used together as an input to the LLM, to make the model generate a more standardized (containing information such as a specific drug name, a specific user intent, etc.) user query (i.e., the rephrased drug question text), ensuring that the rephrased query is semantically the same as the current query. Rephrasing the drug question text helps the system understand the drug question text entered by the user, thereby improving the accuracy of the question answering system.

As an optional implementation, the rephrasing the drug question text includes:
obtaining historical dialog information related to the drug question text, and rephrasing the drug question text based on drug information in the historical dialog information.

Specifically, the present disclosure provides a method for rephrasing the user query. First, the last query of the user is supplemented with the drug information in contextual information of a plurality of rounds of dialogs. For example, in the first round of dialog, the user queries “What is the best drug for treating headaches?”, and after an answer that the drug is ibuprofen is obtained, the current user query “What are the side effects of this drug?” is rephrased into “What are the side effects of ibuprofen?”, thereby improving the accuracy of the question answering system. Second, the user query in the natural language is converted into a machine-understandable form by means of a method such as entity recognition, thereby improving the accuracy of the query.

As an optional implementation, step S103 of performing retrieval in drug instructions in the drug knowledge base based on the drug question text, to obtain instruction text corresponding to the drug question text, as shown in FIG. 3, includes the following steps:

Step S1031: Perform entity recognition on the drug question text, to obtain a drug name corresponding to the drug question text.

Step S1032: Perform retrieval in the drug knowledge base based on the drug name, to obtain a drug instruction corresponding to the drug question text.

Step S1033: Extract, from the drug instruction, the instruction text corresponding to the drug question text.

Specifically, in order to improve the accuracy of the retrieval, it is required to perform entity recognition of the drug mentioned in the query, and a step of the entity recognition can be completed during the query rephrasing. Content irrelevant to the drug is filtered out based on a drug entity, thereby improving the accuracy of extracted content. The entity recognition may be performed using a conventional named entity recognition (NER) technique or by means of a keyword matching method, so as to recognize the drug name. After the drug name is recognized, the retrieval is performed based on the drug name to obtain the corresponding drug instruction, and then the instruction text corresponding to the query is extracted from the drug instruction. For example, the drug question text is “What are the side effects of ibuprofen?”, the entity recognition is performed on the drug question text in step S1031 to obtain the drug name “ibuprofen”, the retrieval in the drug knowledge base is performed based on the drug name “ibuprofen” in step S1032 to obtain a drug instruction of ibuprofen, and then the instruction text relevant to “side effects” is extracted from the drug instruction of ibuprofen in step S1033 to answer the question of the user, thereby improving the accuracy of the question answering system.

As an optional implementation, the performing entity recognition on the drug question text, to obtain a drug name corresponding to the drug question text includes:
scanning a two-dimensional barcode corresponding to a drug package, or performing text recognition based on an obtained image of the drug package, to obtain the drug name corresponding to the drug question text.

The present disclosure allows answering the user query about a drug in a vivid manner with the help of a digital human. For example, the user scans the two-dimensional barcode on the drug package using a mobile phone, and then a digital human is displayed on the mobile phone to give some instructions for use. The user enters a question about the drug in a dialog box, and then the digital human gives an answer, to facilitate use by the user.

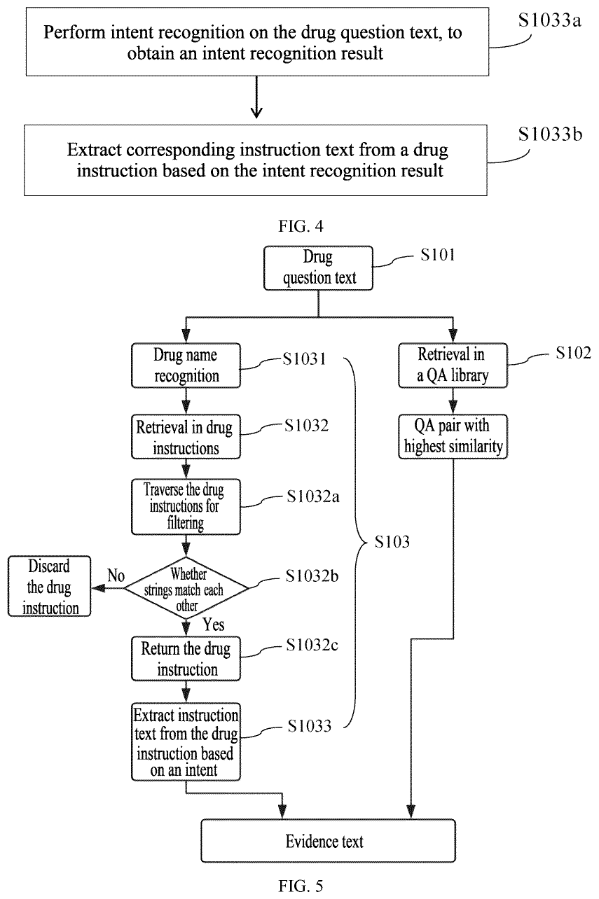

As an optional implementation, step S1033 of extracting, from the drug instruction, the instruction text corresponding to the drug question text, as shown in FIG. 4, includes the following steps:

Step S1033a: Perform intent recognition on the drug question text, to obtain an intent recognition result.

Step S1033b: Extract the corresponding instruction text from the drug instruction based on the intent recognition result.

In the present disclosure, a machine learning technology is used to classify an intent of the user query, where the intent can be classified into a plurality of categories, such as a usage and dosage, an interaction, basic information, an adverse reaction, a contraindication, an effect, and a precaution. The intent recognition is used to determine to which portion of the content in the drug instruction the intent of the user query belongs, and then the corresponding portion is located for retrieval, improving the accuracy and efficiency of the retrieval. For example, when the user queries “What are the side effects of this drug?”, the retrieval is performed in a portion “Adverse Reactions” of the drug instruction.

Specifically, an intent classification model is a general text classification model, such as a convolutional neural network (CNN) model, a recurrent neural network (RNN) model, or a Transformer model, where a softmax layer is connected to an output layer to calculate a cross-entropy loss, so as optimize the model by means of a gradient descent method, thereby completing model training.

As an optional implementation, step S1032 of performing retrieval in the drug knowledge base based on the drug name, to obtain a drug instruction corresponding to the drug question text includes:
calculating similarity based on the drug name corresponding to the drug question text and each of the drug instructions in the drug knowledge base, to obtain the drug instruction with highest similarity.

Step S1033 of extracting, from the drug instruction, the instruction text corresponding to the drug question text includes:
filtering the corresponding instruction text from the drug instruction with the highest similarity based on the intent recognition result.

In this embodiment, in step S103, the drug name may be recognized, a similarity score may be calculated based on the drug name and a drug name in a drug instruction, and then drug instructions are sorted according to similarity scores, so that drug instructions with top 5 similarity scores may be selected. Due to the principle of similarity, a drug in a retrieved drug instruction and the drug in the user query are not necessarily the same drug. Accordingly, the filtering is required. The top 5 drug instructions are traversed to determine whether the drug name in the user query is a substring of the drug name in the drug instruction; if so, it is considered that the drug in the user query and the drug in the drug instruction are the same drug, content in the drug instruction is retained, and then the traversal stops; and if not, the content in the drug instruction is discarded. If the drug instruction is retrieved, the instruction text corresponding to the intent of the query is retained based on the intent.

For example, as shown in FIG. 5, a complete evidence retrieval process in this embodiment includes: after the drug question text is obtained in step S101, retrieving, by the system, evidence text by means of two parallel branches, where a first branch is performing the retrieval in the QA library by using step S102, to obtain the QA pairs with the top 5 similarity scores; and a second branch is performing the retrieval in the drug instructions by using step S103, to obtain the corresponding instruction text.

Step S103 shown in FIG. 5 specifically includes: step S1031: recognizing the drug name; step S1032: retrieving the corresponding drug instruction based on the drug name; and step S1033: extracting the corresponding instruction text from the drug instruction based on the user intent.

Step S1032 shown in FIG. 5 further includes: step S1032a: traversing the top 5 drug instructions to filter the drug instructions; step S1032b: determining whether the drug name in the user query is a substring of the drug name in the drug instruction, if so, considering that the drug in the user query and the drug in the drug instruction are the same drug, retaining content in the drug instruction, and then stopping the traversal; and step S1032c: returning, by the system, the found drug instruction.

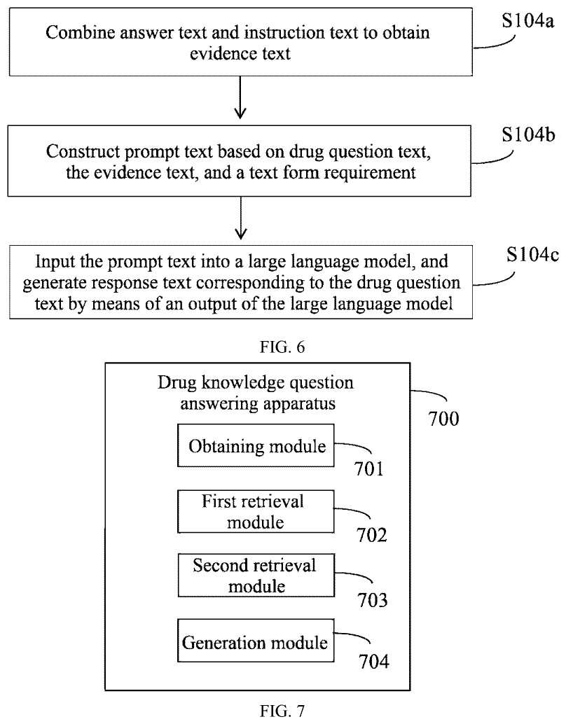

As an optional implementation, step S104 of generating response text corresponding to the drug question text based on the answer text and the instruction text, as shown in FIG. 6, includes the following steps:

Step S104a: Combine the answer text and the instruction text to obtain evidence text.

Step S104b: Construct prompt text (prompt) based on the drug question text, the evidence text, and a text form requirement.

Step S104c: Input the prompt text into a large language model, and generate the response text corresponding to the drug question text by means of an output of the large language model.

After the answer text and the instruction text are respectively retrieved in step S102 and step S103, in this embodiment, the QA pair and the content of the retrieved drug instruction are combined in step S104a, to obtain the final evidence text. Further, the prompt is constructed based on the combined evidence text in step S104b, then the prompt is input into the large language model in step S104c, and the large language model outputs the response text based on the prompt, thereby improving the answering accuracy and the recall of the question answering system.

Specifically, prompt assembly is a skilled step. Proper prompt assembly can reduce hallucinations in LLM answering, improving the answering accuracy. In a drug question answering scenario, the answering accuracy needs to be fully considered to avoid harm to the user. Accordingly, in order to make full use of the ability of the large language model, thought reinforcement logic is constructed relying on a carrier in a json format, and a one-shot mode is introduced to improve the answering accuracy of the large model.

The prompt is constructed in step S104b mainly based on three parts: the rephrased user query, the retrieved evidence text, and the form requirement for text output by the question answering system. A first part includes query rephrasing and drug name recognition, where it is required to determine whether a historical dialog is to be used, a history_use field is true if the answering relies on the historical dialog, and considering a contextual reference, a regenerated question is placed in a query_rephrase field, analysis is placed in a history_use_reason field, and the drug name in the current query is placed in a drug_name field. A second part includes evidence understanding and answering, where answering content is placed in a response field, cited retrieval evidence points are placed in a cite_list list field, a cite_content field stores each paragraph of cited content (in which omitted words can be represented by an ellipsis), a reason_for_cite field is used to store thoughts and analysis for citation of the paragraph of content. If provided reference evidence is irrelevant to the question, the reference evidence is placed in an unrelevant field with a reason explained. The form requirement in a third part may be the one-shot mode, where an overall idea is returning an example corresponding to the above depiction to the large language model, with an example output format as follows:

```
  {
  “history_use”: “true/false”,
  “query_rephrase”: “xxxx”,
  “history_use_reason”: “xxxx”,
  “unrelevant”: “xxxx”,
  “cite_use”: “true/false”,
  “cite_list”: [
  {“cite_content”: “[1]xxxx”, “reason_for_cite”: “xxxx is mentioned in
this paragraph of content, which can answer the question raised”}
  ],
  “response”: “answering content”
  }
```

The above steps include: whether a historical dialog is to be used; what is a regenerated query; regenerating analysis of the query; what is the provided reference evidence irrelevant to the question; whether evidence is cited; which retrieval evidence points are included in the list; what is the cited evidence; thoughts and analysis for citation of the paragraph of content; and answering for the query. The prompt assembly enables the large language model to better understand the question of the user and generate more understandable text to answer the question of the user.

As an optional implementation, before step S102 of performing retrieval in question-answer text in a drug knowledge base based on the drug question text, the method further includes:
parsing question answering data into the question-answer text, and storing the question-answer text in the drug knowledge base.

Specifically, before use of the question answering system, a database needs to be preprocessed to obtain the drug knowledge base. The present disclosure allows structured processing on the drug instruction using the natural language processing technology, to extract key information such as the name, components, indications, usage and dosage, and adverse reactions of the drug and store the key information in the database. Specifically, the unstructured drug instruction text is subjected to the structured processing based on keywords such as the drug name and drug components, and a rule such as a text structure of these keywords (for example, location in a first line of a paragraph), and then stored in the database. In addition, in order to enhance a generalization capability of the question answering system, the question answering data is uniformly parsed into <question, answer> pairs (i.e., QA pairs), which are stored in the database to construct the drug knowledge base.

The present disclosure further provides a drug knowledge question answering apparatus 700. As shown in FIG. 7, the apparatus includes:
an obtaining module 701 configured to obtain drug question text;
a first retrieval module 702 configured to perform retrieval in question-answer text in a drug knowledge base based on the drug question text, to obtain answer text corresponding to the drug question text;
a second retrieval module 703 configured to perform retrieval in drug instructions in the drug knowledge base based on the drug question text, to obtain instruction text corresponding to the drug question text; and
a generation module 704 configured to generate response text corresponding to the drug question text based on the answer text and the instruction text.

Specifically, the drug question text in this embodiment refers to a query entered by the user, such as a question “What is the best drug for treating headaches?”, “What are the side effects of this drug?”, etc. The question-answer text refers to QA pairs, where each QA pair includes a question and a corresponding answer. For example, for a question “What are the side effects of ibuprofen?”, a corresponding answer is “The side effects of ibuprofen may be gastrointestinal discomfort including nausea, vomiting, abdominal pain, burning sensation or mild indigestion, or gastrointestinal ulcers and bleeding, and sometimes may cause a rash, urticaria, etc.”. The first retrieval module 702 may perform QA pair matching first, and if a corresponding question Q is found, directly return a corresponding answer A as the answer text.

If the first retrieval module 702 finds no answer text, the second retrieval module 703 may perform drug instruction matching to find a drug instruction corresponding to a drug name contained in the query, then retrieve relevant drug knowledge from the corresponding drug instruction, and return it to the user. The drug instruction usually includes much content, and the retrieval may be performed to return only drug knowledge relevant to the query to the user. For example, if the user enters “What are the side effects of this drug?”, only a paragraph relevant to the side effects in the drug instruction is to be extracted as the instruction text, so as to implement accurate matching.

The present disclosure allows a combination of the retrieval in the question-answer text and retrieval in drug instruction paragraphs, where the answer related to the user query may be quickly and accurately retrieved by the retrieval in the question-answer text, and when no accurate answer is found, the retrieval in the drug instruction paragraphs may be performed as a supplement, so as to ensure the recall of the question answering system, improving the answering accuracy and the recall of the system.

As an optional implementation, in performing, by a first retrieval module 702, retrieval in question-answer text in a drug knowledge base based on the drug question text, to obtain answer text corresponding to the drug question text, a conventional retrieval algorithm, such as a TF-IDF algorithm, may be selected. Alternatively, a vector retrieval method may be selected, where most relevant content is recalled by calculating cosine similarity etc. between a user query vector and a drug instruction content vector. For example, similarity is calculated based on a query and a question Q in a QA library, then QA pairs are sorted according to similarity scores, and QA pairs with top 5 similarity scores are selected, thereby implementing the accurate matching for the question of the user.

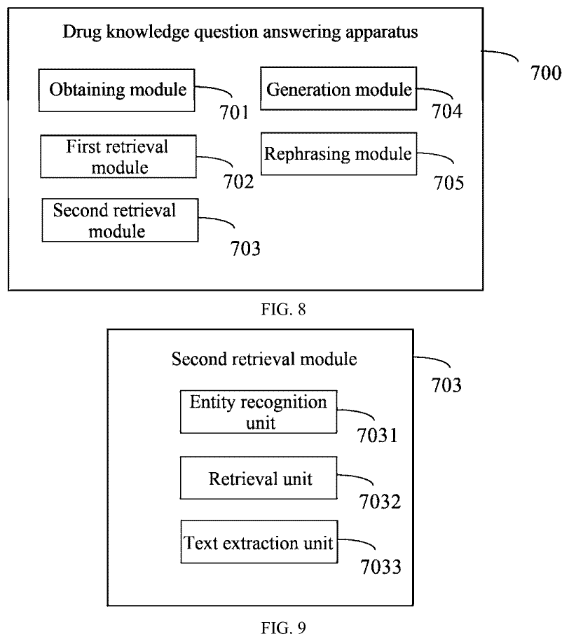

As an optional implementation, as shown in FIG. 8, the drug knowledge question answering apparatus 700 further includes:
a rephrasing module 705 configured to rephrase the drug question text before the retrieval in the question-answer text in the drug knowledge base is performed based on the drug question text.

Specifically, users may use various languages and expressions to describe their needs during queries for drug information. For example, the query may be “What is the best drug for treating headaches?”, “What are the side effects of this drug?”, etc. In a classical natural language processing technology, it is required to perform an operation, such as word segmentation or stop word removal, on the user query, or to rephrase the user query according to a preset rule. In this embodiment, the above operation may be implemented with the help of a prompting technique of a large language model. Specifically, contextual historical dialog information of user queries and a current question entered by the user are assembled into a complete contextual dialog, and then dialog content and a prompt requirement are used together as an input to the LLM, to make the model generate a more standardized (containing information such as a specific drug name, a specific user intent, etc.) user query (i.e., the rephrased drug question text), ensuring that the rephrased query is semantically the same as the current query. Rephrasing the drug question text helps the system understand the drug question text entered by the user, thereby improving the accuracy of the question answering system.

As an optional implementation, the rephrasing, by a rephrasing module 705, the drug question text includes:
obtaining historical dialog information related to the drug question text, and rephrasing the drug question text based on drug information in the historical dialog information.

Specifically, the present disclosure provides a method for rephrasing the user query. First, the last query of the user is supplemented with the drug information in contextual information of a plurality of rounds of dialogs. For example, in the first round of dialog, the user queries “What is the best drug for treating headaches?”, and after an answer that the drug is ibuprofen is obtained, the current user query “What are the side effects of this drug?” is rephrased into “What are the side effects of ibuprofen?”, thereby improving the accuracy of the question answering system. Second, the user query in the natural language is converted into a machine-understandable form by means of a method such as entity recognition, thereby improving the accuracy of the query.

As an optional implementation, as shown in FIG. 9, the second retrieval module 703 includes:
an entity recognition unit 7031 configured to perform entity recognition on the drug question text, to obtain a drug name corresponding to the drug question text;
a retrieval unit 7032 configured to perform retrieval in the drug knowledge base based on the drug name, to obtain a drug instruction corresponding to the drug question text; and
a text extraction unit 7033 configured to extract, from the drug instruction, the instruction text corresponding to the drug question text.

Specifically, in order to improve the accuracy of the retrieval, it is required to perform entity recognition of the drug mentioned in the query, and a step of the entity recognition can be completed during the query rephrasing. Content irrelevant to the drug is filtered out based on a drug entity, thereby improving the accuracy of extracted content. The entity recognition may be performed using a conventional NER technique or by means of a keyword matching method, so as to recognize the drug name. After the drug name is recognized, the retrieval is performed based on the drug name to obtain the corresponding drug instruction, and then the instruction text corresponding to the query is extracted from the drug instruction. For example, the drug question text is “What are the side effects of ibuprofen?”, the entity recognition unit 7031 performs the entity recognition on the drug question text to obtain the drug name “ibuprofen”, the retrieval unit 7032 performs the retrieval in the drug knowledge base based on the drug name “ibuprofen” to obtain a drug instruction of ibuprofen, and then the text extraction unit 7033 extracts the instruction text relevant to “side effects” from the drug instruction of ibuprofen to answer the question of the user, thereby improving the accuracy of the question answering system.

As an optional implementation, the performing, by an entity recognition unit 7031, entity recognition on the drug question text, to obtain a drug name corresponding to the drug question text includes:
scanning a two-dimensional barcode corresponding to a drug package, or performing text recognition based on an obtained image of the drug package, to obtain the drug name corresponding to the drug question text.

The present disclosure allows answering the user query about a drug in a vivid manner with the help of a digital human. For example, the user scans the two-dimensional barcode on the drug package using a mobile phone, and then a digital human is displayed on the mobile phone to give some instructions for use. The user enters a question about the drug in a dialog box, and then the digital human gives an answer.

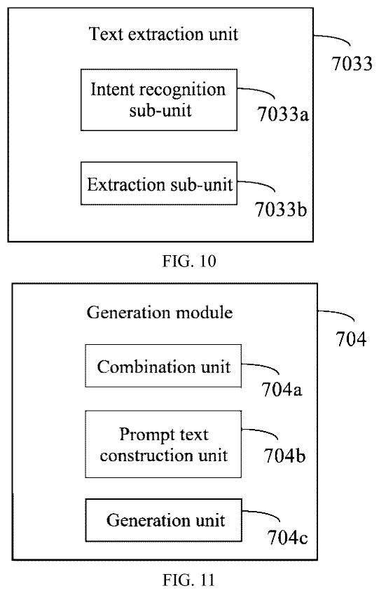

As an optional implementation, as shown in FIG. 10, the text extraction unit 7033 includes:
an intent recognition sub-unit 7033a configured to perform intent recognition on the drug question text, to obtain an intent recognition result; and
an extraction sub-unit 7033b configured to extract the corresponding instruction text from the drug instruction based on the intent recognition result.

In the present disclosure, a machine learning technology is used to classify an intent of the user query, where the intent can be classified into a plurality of categories, such as a usage and dosage, an interaction, basic information, an adverse reaction, a contraindication, an effect, and a precaution. The intent recognition is used to determine to which portion of the content in the drug instruction the intent of the user query belongs, and then the corresponding portion is located for retrieval. For example, when the user queries “What are the side effects of this drug?”, the retrieval is performed in a portion “Adverse Reactions” of the drug instruction.

Specifically, an intent classification model may be a general text classification model, such as a CNN model, an RNN model, or a Transformer model, where a softmax layer is connected to an output layer to calculate a cross-entropy loss, so as optimize the model by means of a gradient descent method, thereby completing model training.

As an optional implementation, the performing, by a retrieval unit 7032, retrieval in the drug knowledge base based on the drug name, to obtain a drug instruction corresponding to the drug question text includes:
calculating similarity based on the drug name corresponding to the drug question text and each of the drug instructions in the drug knowledge base, to obtain the drug instruction with highest similarity.

The extracting, by a text extraction unit 7033 from the drug instruction, the instruction text corresponding to the drug question text includes:
filtering the corresponding instruction text from the drug instruction with the highest similarity based on the intent recognition result.

In this embodiment, the drug name may be recognized, a similarity score may be calculated based on the drug name and a drug name in a drug instruction, and then drug instructions are sorted according to similarity scores, so that drug instructions with top 5 similarity scores may be selected by the retrieval unit 7032. Due to the principle of similarity, a drug in a retrieved drug instruction and the drug in the user query are not necessarily the same drug. Accordingly, the filtering is required. The top 5 drug instructions are traversed to determine whether the drug name in the user query is a substring of the drug name in the drug instruction; if so, it is considered that the drug in the user query and the drug in the drug instruction are the same drug, content in the drug instruction is retained, and then the traversal stops; and if not, the content in the drug instruction is discarded. If the drug instruction is retrieved, the text extraction unit 7033 retains the instruction text corresponding to the intent of the query based on the intent.

As an optional implementation, as shown in FIG. 11, the generation module 704 includes:
a combination unit 704a configured to combine the answer text and the instruction text to obtain evidence text;
a prompt text construction unit 704b configured to construct prompt text (prompt) based on the drug question text, the evidence text, and a text form requirement; and
a generation unit 704c configured to input the prompt text into a large language model, and generate the response text corresponding to the drug question text by means of an output of the large language model.

After the answer text and the instruction text are respectively retrieved by the first retrieval module 702 and the second retrieval module 703, in this embodiment, the system combines the QA pair and the content of the retrieved drug instruction by means of the combination unit 704a, to obtain the final evidence text. Further, the prompt text construction unit 704b constructs the prompt based on the combined evidence text, then the generation unit 704c inputs the prompt into the large language model, and the large language model outputs the response text based on the prompt, thereby improving the answering accuracy and the recall of the question answering system.

Specifically, prompt assembly is a skilled step. Proper prompt assembly can reduce hallucinations in LLM answering, improving the answering accuracy. In a drug question answering scenario, the answering accuracy needs to be fully considered to avoid harm to the user. Accordingly, in order to make full use of the ability of the large language model, thought reinforcement logic is constructed relying on a carrier in a json format, and a one-shot mode is introduced to improve the answering accuracy of the large model.

A specific prompt construction method is mainly based on three parts: the rephrased user query, the retrieved evidence text, and the form requirement for text output by the question answering system. A first part includes query rephrasing and drug name recognition, where it is required to determine whether a historical dialog is to be used, a history_use field is true if the answering relies on the historical dialog, and considering a contextual reference, a regenerated question is placed in a query_rephrase field, analysis is placed in a history_use_reason field, and the drug name in the current query is placed in a drug_name field. A second part includes evidence understanding and answering, where answering content is placed in a response field, cited retrieval evidence points are placed in a cite_list list field, a cite_content field stores each paragraph of cited content (in which omitted words can be represented by an ellipsis), a reason_for_cite field is used to store thoughts and analysis for citation of the paragraph of content. If provided reference evidence is irrelevant to the question, the reference evidence is placed in an unrelevant field with a reason explained. The form requirement in a third part may be the one-shot mode, where an overall idea is returning an example corresponding to the above depiction to the large language model, with an example output format as follows:

```
  {
  “history_use”: “true/false”,
  “query_rephrase”: “xxxx”,
  “history_use_reason”: “xxxx”,
  “unrelevant”: “xxxx”,
  “cite_use”: “true/false”,
  “cite_list”: [
  {“cite_content”: “[1]xxxx”, “reason_for_cite”: “xxxx is mentioned in
this paragraph of content, which can answer the question raised”}
  ],
  “response”: “answering content”
  }
```

The above steps include: whether a historical dialog is to be used; what is a regenerated query; regenerating analysis of the query; what is the provided reference evidence irrelevant to the question; whether evidence is cited; which retrieval evidence points are included in the list; what is the cited evidence; thoughts and analysis for citation of the paragraph of content; and answering for the query. The prompt assembly enables the large language model to better understand the question of the user and generate more understandable text to answer the question of the user.

As an optional implementation, the drug knowledge question answering apparatus 700 further includes:
a data processing module configured to, before the first retrieval module 702 performs the retrieval in the question-answer text in the drug knowledge base based on the drug question text, parse question answering data into the question-answer text, and store the question-answer text in the drug knowledge base.

Specifically, before use of the question answering system, a database needs to be preprocessed to obtain the drug knowledge base. The present disclosure allows structured processing on the drug instruction using the natural language processing technology, to extract key information such as the name, components, indications, usage and dosage, and adverse reactions of the drug and store the key information in the database. Specifically, the unstructured drug instruction text is subjected to the structured processing based on keywords such as the drug name and drug components, and a rule such as a text structure of these keywords (for example, location in a first line of a paragraph), and then stored in the database. In addition, in order to enhance a generalization capability of the question answering system, the question answering data is uniformly parsed into <question, answer> pairs (i.e., QA pairs), which are stored in the database to construct the drug knowledge base.

In the technical solutions of the present disclosure, obtaining, storage, application, etc. of personal information of a user all comply with related laws and regulations and are not against the public order and good morals.

According to an embodiment of the present disclosure, the present disclosure further provides an electronic device, a readable storage medium, and a computer program product.

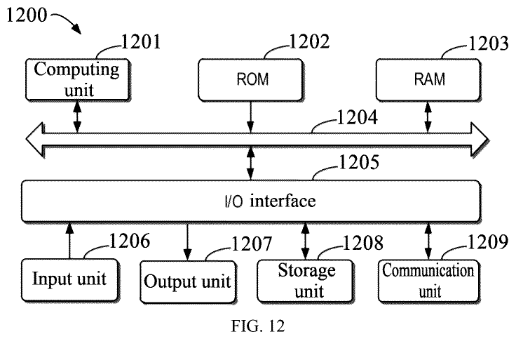

FIG. 12 is a schematic block diagram of an exemplary electronic device 1200 that can be used to implement an embodiment of the present disclosure. The electronic device is intended to represent various forms of digital computers, such as a laptop computer, a desktop computer, a workstation, a personal digital assistant, a server, a blade server, a mainframe computer, and other suitable computers. The electronic device may further represent various forms of mobile apparatuses, such as a personal digital assistant, a cellular phone, a smartphone, a wearable device, and other similar computing apparatuses. The components shown in the present specification, their connections and relationships, and their functions are merely examples, and are not intended to limit the implementation of the present disclosure described and/or required herein.

As shown in FIG. 12, the device 1200 includes a computing unit 1201. The computing unit may perform various appropriate actions and processing according to a computer program stored in a read-only memory (ROM) 1202 or a computer program loaded from a storage unit 1208 to a random access memory (RAM) 1203. The RAM 1203 may further store various programs and data required for the operation of the device 1200. The computing unit 1201, the ROM 1202, and the RAM 1203 are connected to each other through a bus 1204. An input/output (I/O) interface 1205 is also connected to the bus 1204.

A plurality of components in the device 1200 are connected to the I/O interface 1205, including: an input unit 1206, such as a keyboard or a mouse; an output unit 1207, such as various types of displays or speakers; a storage unit 1208, such as a magnetic disk or an optical disk; and a communication unit 1209, such as a network interface card, a modem or a wireless communications transceiver. The communication unit 1209 allows the device 1200 to exchange information/data with other devices via a computer network such as the Internet and/or various telecommunications networks.

The computing unit 1201 may be various general-purpose and/or special-purpose processing components with processing and computing capabilities. Some examples of the computing unit 1201 include, but are not limited to, a central processing unit (CPU), a graphics processing unit (GPU), various dedicated artificial intelligence (AI) computing chips, various computing units that run machine learning objective function algorithms, a digital signal processor (DSP), and any appropriate processor, controller, microcontroller, etc. The computing unit 1201 performs the various methods and processing described above, for example, the drug knowledge question answering method. For example, in some embodiments, the drug knowledge question answering method may be implemented as a computer software program, which is tangibly contained in a machine-readable medium, such as the storage unit 1208. In some embodiments, a part or all of the computer program may be loaded and/or installed onto the device 1200 via the ROM 1202 and/or the communication unit 1209. When the computer program is loaded onto the RAM 1203 and executed by the computing unit 1201, one or more steps of the drug knowledge question answering method described above can be performed. Alternatively, in other embodiments, the computing unit 1201 may be configured, by any other appropriate means (for example, by means of firmware), to perform the drug knowledge question answering method.

Various implementations of the systems and technologies described herein above can be implemented in a digital electronic circuit system, an integrated circuit system, a field programmable gate array (FPGA), an application-specific integrated circuit (ASIC), an application-specific standard product (ASSP), a system-on-chip (SOC) system, a complex programmable logical device (CPLD), computer hardware, firmware, software, and/or a combination thereof. These various implementations may include: implementation in one or more computer programs, where the one or more computer programs may be executed and/or interpreted on a programmable system including at least one programmable processor. The programmable processor may be a dedicated or general-purpose programmable processor that can receive data and instructions from a storage system, at least one input apparatus, and at least one output apparatus, and transmit data and instructions to the storage system, the at least one input apparatus, and the at least one output apparatus.

Program codes used to implement the method of the present disclosure can be written in any combination of one or more programming languages. These program codes may be provided for a processor or a controller of a general-purpose computer, a special-purpose computer, or other programmable data processing apparatuses, such that when the program codes are executed by the processor or the controller, the functions/operations specified in the flowcharts and/or block diagrams are implemented. The program codes may be completely executed on a machine, or partially executed on a machine, or may be, as an independent software package, partially executed on a machine and partially executed on a remote machine, or completely executed on a remote machine or a server.

In the context of the present disclosure, the machine-readable medium may be a tangible medium, which may contain or store a program for use by an instruction execution system, apparatus, or device, or for use in combination with the instruction execution system, apparatus, or device. The machine-readable medium may be a machine-readable signal medium or a machine-readable storage medium. The machine-readable medium may include, but is not limited to, an electronic, magnetic, optical, electromagnetic, infrared, or semiconductor system, apparatus, or device, or any suitable combination thereof. More specific examples of the machine-readable storage medium may include an electrical connection based on one or more wires, a portable computer disk, a hard disk, a random access memory (RAM), a read-only memory (ROM), an erasable programmable read-only memory (EPROM or flash memory), an optical fiber, a portable compact disk read-only memory (CD-ROM), an optical storage device, a magnetic storage device, or any suitable combination thereof.

In order to provide interaction with a user, the systems and technologies described herein can be implemented on a computer which has: a display apparatus (for example, a cathode-ray tube (CRT) or a liquid crystal display (LCD) monitor) configured to display information to the user; and a keyboard and a pointing apparatus (for example, a mouse or a trackball) through which the user can provide an input to the computer. Other categories of apparatuses can also be used to provide interaction with the user; for example, feedback provided to the user can be any form of sensory feedback (for example, visual feedback, auditory feedback, or tactile feedback), and an input from the user can be received in any form (including an acoustic input, a voice input, or a tactile input).

The systems and technologies described herein can be implemented in a computing system (for example, as a data server) including a backend component, or a computing system (for example, an application server) including a middleware component, or a computing system (for example, a user computer with a graphical user interface or a web browser through which the user can interact with the implementation of the systems and technologies described herein) including a frontend component, or a computing system including any combination of the backend component, the middleware component, or the frontend component. The components of the system can be connected to each other through digital data communication (for example, a communication network) in any form or medium. Examples of the communication network include: a local area network (LAN), a wide area network (WAN), and the Internet.

A computer system may include a client and a server. The client and the server are generally far away from each other and usually interact through a communication network. A relationship between the client and the server is generated by computer programs running on respective computers and having a client-server relationship with each other. The server may be a cloud server, a server in a distributed system, or a server combined with a blockchain.

It should be understood that steps may be reordered, added, or deleted based on the various forms of procedures shown above. For example, the steps recorded in the present disclosure may be performed in parallel, in order, or in a different order, provided that the desired result of the technical solutions disclosed in the present disclosure can be achieved, which is not limited herein.

The specific implementations above do not constitute a limitation on the protection scope of the present disclosure. Those skilled in the art should understand that various modifications, combinations, sub-combinations, and replacements can be made based on design requirements and other factors. Any modifications, equivalent replacements, improvements, etc. within the spirit and principle of the present disclosure shall fall within the protection scope of the present disclosure.

What is claimed is:

1. A drug knowledge question answering method, comprising:
obtaining drug question text;
performing retrieval in question-answer text in a drug knowledge base based on the drug question text, to obtain answer text corresponding to the drug question text;
performing retrieval in drug instructions in the drug knowledge base based on the drug question text, to obtain instruction text corresponding to the drug question text; and
generating response text corresponding to the drug question text based on the answer text and the instruction text.

2. The method according to claim 1, wherein before the performing retrieval in question-answer text in a drug knowledge base based on the drug question text, the method further comprises:
rephrasing the drug question text.

3. The method according to claim 2, wherein the rephrasing the drug question text comprises:
obtaining historical dialog information related to the drug question text; and
rephrasing the drug question text based on drug information in the historical dialog information.

4. The method according to claim 1, wherein the performing retrieval in drug instructions in the drug knowledge base based on the drug question text, to obtain instruction text corresponding to the drug question text comprises:
performing entity recognition on the drug question text, to obtain a drug name corresponding to the drug question text;
performing retrieval in the drug knowledge base based on the drug name, to obtain a drug instruction corresponding to the drug question text; and
extracting, from the drug instruction, the instruction text corresponding to the drug question text.

5. The method according to claim 4, wherein the performing entity recognition on the drug question text, to obtain a drug name corresponding to the drug question text comprises:
scanning a two-dimensional barcode corresponding to a drug package, or performing text recognition based on an obtained image of the drug package, to obtain the drug name corresponding to the drug question text.

6. The method according to claim 4, wherein the extracting, from the drug instruction, the instruction text corresponding to the drug question text comprises:
performing intent recognition on the drug question text, to obtain an intent recognition result; and
extracting the instruction text from the drug instruction based on the intent recognition result.

7. The method according to claim 6, wherein
the performing retrieval in the drug knowledge base based on the drug name, to obtain a drug instruction corresponding to the drug question text comprises:
calculating similarity based on the drug name corresponding to the drug question text and each of the drug instructions in the drug knowledge base, to obtain the drug instruction with the highest similarity; and
the extracting, from the drug instruction, the instruction text corresponding to the drug question text comprises:
extracting the instruction text from the drug instruction with the highest similarity based on the intent recognition result.

8. The method according to claim 1, wherein the generating response text corresponding to the drug question text based on the answer text and the instruction text comprises:
combining the answer text and the instruction text to obtain evidence text;
constructing prompt text based on the drug question text, the evidence text, and a text form requirement; and
generating the response text corresponding to the drug question text by means of an output of a large language model by inputting the prompt text into the large language model.

9. The method according to claim 1, wherein before the performing retrieval in question-answer text in a drug knowledge base based on the drug question text, the method further comprises:
parsing question answering data into the question-answer text, and storing the question-answer text in the drug knowledge base.

10. An electronic device, comprising:
at least one processor; and
a memory communicatively connected to the at least one processor, wherein
the memory stores instructions executable by the at least one processor, and the instructions, when executed by the at least one processor, cause the at least one processor to perform the following steps:
obtaining drug question text;
performing retrieval in question-answer text in a drug knowledge base based on the drug question text, to obtain answer text corresponding to the drug question text;
performing retrieval in drug instructions in the drug knowledge base based on the drug question text, to obtain instruction text corresponding to the drug question text; and
generating response text corresponding to the drug question text based on the answer text and the instruction text.

11. The electronic device according to claim 10, wherein before the performing retrieval in question-answer text in a drug knowledge base based on the drug question text, the following step is further comprised:
rephrasing the drug question text.

12. The electronic device according to claim 11, wherein the rephrasing the drug question text comprises:
obtaining historical dialog information related to the drug question text; and
rephrasing the drug question text based on drug information in the historical dialog information.

13. The electronic device according to claim 10, wherein the performing retrieval in drug instructions in the drug knowledge base based on the drug question text, to obtain instruction text corresponding to the drug question text comprises:
performing entity recognition on the drug question text, to obtain a drug name corresponding to the drug question text;
performing retrieval in the drug knowledge base based on the drug name, to obtain a drug instruction corresponding to the drug question text; and
extracting, from the drug instruction, the instruction text corresponding to the drug question text.

14. The electronic device according to claim 13, wherein the performing entity recognition on the drug question text, to obtain a drug name corresponding to the drug question text comprises:
scanning a two-dimensional barcode corresponding to a drug package, or performing text recognition based on an obtained image of the drug package, to obtain the drug name corresponding to the drug question text.

15. The electronic device according to claim 13, wherein the extracting, from the drug instruction, the instruction text corresponding to the drug question text comprises:
performing intent recognition on the drug question text, to obtain an intent recognition result; and
extracting the instruction text from the drug instruction based on the intent recognition result.

16. The electronic device according to claim 15, wherein
the performing retrieval in the drug knowledge base based on the drug name, to obtain a drug instruction corresponding to the drug question text comprises:
calculating similarity based on the drug name corresponding to the drug question text and each of the drug instructions in the drug knowledge base, to obtain the drug instruction with the highest similarity; and
the extracting, from the drug instruction, the instruction text corresponding to the drug question text comprises:
extracting the instruction text from the drug instruction with the highest similarity based on the intent recognition result.

17. The electronic device according to claim 10, wherein the generating response text corresponding to the drug question text based on the answer text and the instruction text comprises:
combining the answer text and the instruction text to obtain evidence text;
constructing prompt text based on the drug question text, the evidence text, and a text form requirement; and
generating the response text corresponding to the drug question text by means of an output of a large language model by inputting the prompt text into the large language model.

18. The electronic device according to claim 10, wherein before the performing retrieval in question-answer text in a drug knowledge base based on the drug question text, the following step is further comprised:
parsing question answering data into the question-answer text, and storing the question-answer text in the drug knowledge base.

19. A non-transitory computer-readable storage medium storing computer instructions, wherein the computer instructions are used to cause the computer to perform the following steps:
obtaining drug question text;
performing retrieval in question-answer text in a drug knowledge base based on the drug question text, to obtain answer text corresponding to the drug question text;
performing retrieval in drug instructions in the drug knowledge base based on the drug question text, to obtain instruction text corresponding to the drug question text; and
generating response text corresponding to the drug question text based on the answer text and the instruction text.

20. The non-transitory computer-readable storage medium according to claim 19, wherein the generating response text corresponding to the drug question text based on the answer text and the instruction text comprises:
combining the answer text and the instruction text to obtain evidence text;
constructing prompt text based on the drug question text, the evidence text, and a text form requirement; and
generating the response text corresponding to the drug question text by means of an output of a large language model by inputting the prompt text into the large language model.
